(19) United States
(12) Patent Application Publication
(10) Pub. No.: US 2003/0228368 A1
(43) Pub. Date: Dec. 11, 2003

(54) Edible solid composition and dosage form

(76) Inventors: David Wynn, Abington, PA (US); Shun-Por Li, Lansdale, PA (US); Narendra Parikh, Long Valley, NJ (US); Harry S. Sowden, Glenside, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/393,756

(22) Filed: Mar. 21, 2003

Related U.S. Application Data
(63) Continuation-in-part of application No. PCT/US02/31129, filed on Sep. 28, 2002.
(63) Continuation-in-part of application No. PCT/US02/31117, filed on Sep. 28, 2002.
(63) Continuation-in-part of application No. PCT/US02/31062, filed on Sep. 28, 2002.
(63) Continuation-in-part of application No. PCT/US02/31024, filed on Sep. 28, 2002.
(63) Continuation-in-part of application No. PCT/US02/31163, filed on Sep. 28, 2002.
Application No. PCT/US02/31129 is a continuation-in-part of application No. 09/966,509, filed on Sep. 28, 2001.
Application No. PCT/US02/31129 is a continuation-in-part of application No. 09/966,497, filed on Sep. 28, 2001.
Application No. PCT/US02/31129 is a continuation-in-part of application No. 09/967,414, filed on Sep. 28, 2001.
Application No. PCT/US02/31129 is a continuation-in-part of application No. 09/966,450, filed on Sep. 28, 2001.
Application No. PCT/US02/31117 is a continuation-in-part of application No. 09/966,509, filed on Sep. 28, 2001.
Application No. PCT/US02/31117 is a continuation-in-part of application No. 09/966,497, filed on Sep. 28, 2001.
Application No. PCT/US02/31117 is a continuation-in-part of application No. 09/967,414, filed on Sep. 28, 2001.
Application No. PCT/US02/31117 is a continuation-in-part of application No. 09/966,450, filed on Sep. 28, 2001.
Application No. PCT/US02/31062 is a continuation-in-part of application No. 09/966,509, filed on Sep. 28, 2001.
Application No. PCT/US02/31062 is a continuation-in-part of application No. 09/966,497, filed on Sep. 28, 2001.
Application No. PCT/US02/31062 is a continuation-in-part of application No. 09/967,414, filed on Sep. 28, 2001.
Application No. PCT/US02/31062 is a continuation-in-part of application No. 09/966,450, filed on Sep. 28, 2001.
Application No. PCT/US02/31024 is a continuation-in-part of application No. 09/966,509, filed on Sep. 28, 2001.
Application No. PCT/US02/31024 is a continuation-in-part of application No. 09/966,497, filed on Sep. 28, 2001.
Application No. PCT/US02/31024 is a continuation-in-part of application No. 09/967,414, filed on Sep. 28, 2001.
Application No. PCT/US02/31024 is a continuation-in-part of application No. 09/966,450, filed on Sep. 28, 2001.
Application No. PCT/US02/31163 is a continuation-in-part of application No. 09/966,509, filed on Sep. 28, 2001.
Application No. PCT/US02/31163 is a continuation-in-part of application No. 09/966,497, filed on Sep. 28, 2001.
Application No. PCT/US02/31163 is a continuation-in-part of application No. 09/967,414, filed on Sep. 28, 2001.
Application No. PCT/US02/31163 is a continuation-in-part of application No. 09/966,450, filed on Sep. 28, 2001.

(51) Int. Cl. A61K 9/14

(52) U.S. Cl. 424/486

(57) ABSTRACT

An edible solid composition comprises: (a) about 25 to about 40 weight percent of at least one non-aqueous carrier material which has a melting temperature less than about 45 degrees C.; and (b) about 15 to about 60 weight percent of at least one thermoplastic material which has a melting temperature greater than about 50 degrees C. The composition may optionally additionally contain up to about 40 weight percent of at least one compatibility material for retaining the non-aqueous carrier material in the edible solid composition. The compatibility material is selected from the group consisting of emulsifiers, acrylic polymers, waxes and combinations thereof. The edible solid composition may be used as a core or shell in a dosage form, or as a dosage form per se which contains or is prepared from such an edible solid composition.

### CROSS-REFERENCE TO RELATED APPLICATIONS

This is a continuation-in-part of PCT Application Nos. PCT/US02/31129, filed Sep. 28, 2002; PCT/US02/31117, filed Sep. 28, 2002; PCT/US02/31062, filed Sep. 28, 2002; PCT/US02/31024, filed Sep. 28, 2002; and PCT/US02/31163, filed Sep. 28, 2002, which are each continuations-in-part of U.S. Ser. No. 09/966,939, filed Sep. 28, 2001; U.S. Ser. No. 09/966,509, filed Sep. 28, 2001; U.S. Ser. No. 09/966,497, filed Sep. 28, 2001; U.S. Ser. No. 09/967,414, filed Sep. 28, 2001; and U.S. Ser. No. 09/966,450, filed Sep. 28, the disclosures of all of the above being incorporated herein by reference in their entirety.

### BACKGROUND OF THE INVENTION

1. Field of the Invention

This invention is directed to an edible solid composition, a core or shell for use in a dosage form such as a pharmaceutical composition, a dosage form per se, and methods of preparing such compositions. More particularly, this invention relates to an edible solid composition containing at least one non-aqueous carrier material which has a melting temperature of less than about 45 degrees C. and at least one thermoplastic material which has a melting temperature greater than about 50 degrees C., as well as cores or shells for use in a dosage form, or dosage forms per se which contain or are prepared from such an edible solid composition.

2. Background Information

Modified release pharmaceutical dosage forms have long been used to optimize drug delivery and enhance patient compliance, especially by reducing the number of doses of medicine the patient must take in a day. For this purpose, it is often desirable to modify the rate of release of a drug (one particularly preferred type of active ingredient) from a dosage form into the GI fluids of a patient, especially to slow the release to provide prolonged action of the drug in the body.

The rate at which an orally delivered pharmaceutical active ingredient reaches its site of action in the body depends on a number of factors, including the rate and extent of drug absorption through the GI mucosa. To be absorbed into the circulatory system (blood), the drug must first be dissolved in the GI fluids. For many drugs, diffusion across the GI membranes is relatively rapid compared to dissolution. In these cases, the dissolution of the active ingredient is the rate limiting step in drug absorption, and controlling the rate of dissolution allows the formulator to control the rate of drug absorption into the circulatory system of a patient.

An important objective of modified release dosage forms is to provide a desired blood concentration versus time (pharmacokinetic, or PK) profile for the drug. Fundamentally, the PK profile for a drug is governed by the rate of absorption of the drug into the blood, and the rate of elimination of the drug from the blood. The type of PK profile desired depends, among other factors, on the particular active ingredient, and physiological condition being treated.

A particularly desirable PK profile for a number of drugs and conditions is one in which the level of drug in the blood is maintained essentially constant (i.e. the rate of drug absorption is approximately equal to the rate of drug elimination) over a relatively long period of time. Such systems have the benefit of reducing the frequency of dosing, improving patient compliance, as well as minimizing side effects while maintaining full therapeutic efficacy. A dosage form which provides a “zero-order,” or constant release rate of the drug is useful for this purpose. Since zero-order release systems are difficult to achieve, systems which approximate a constant release rate, such as for example first-order and square root of time profiles are often used to provide sustained (e.g. prolonged, extended, or retarded) release of a drug.

It is also particularly desirable for a pharmaceutical dosage form to deliver more than one drug at a modified rate. Because the onset and duration of the therapeutic efficacy of drugs vary widely, as do their absorption, distribution, metabolism, and elimination, it is often desirable to modify the release of different drugs in different ways, or to have a first active ingredient immediately released from the dosage form, while a second drug is released in a delayed, controlled, sustained, prolonged, extended, or retarded manner. Modified release dosage forms should ideally be adaptable so that release rates and profiles can be matched to physiological and chronotherapeutic requirements.

Well known mechanisms by which a dosage form (or drug delivery system) can deliver drug at a controlled rate (e.g. sustained, prolonged, extended or retarded release) include diffusion, erosion, and osmosis.

One classic diffusion-controlled release system comprises a “reservoir” containing the active ingredient, surrounded by a “membrane” through which the active ingredient must diffuse to be absorbed into the bloodstream of the patient. The rate of drug release, dM/dt depends on the area (A) of the membrane, the diffusional pathlength (1), the concentration gradient (DC) of the drug across the membrane, the partition coefficient (K) of the drug into the membrane, and the diffusion coefficient (D) according to the following equation:

dM/dt={ADKΔC}/1

Since one or more of the above terms, particularly the diffusional pathlength, and concentration gradient tend to be non-constant, diffusion-controlled systems generally deliver a non-constant release rate. In general, the rate of drug release from diffusion-controlled release systems typically follows first order kinetics.

Another common type of diffusion-controlled release system comprises active ingredient, distributed throughout an insoluble porous matrix through which the active ingredient must diffuse to be absorbed into the bloodstream of the patient. The amount of drug release (M) at a given time at sink conditions (i.e. drug concentration at the matrix surface is much greater than drug concentration in the bulk solution) depends on the area (A) of the matrix, the diffusion coefficient (D), the porosity (E) and tortuosity (T) of the matrix, the drug solubility (Cs) in the dissolution medium, time (t) and the drug concentration (Cp) in the dosage form according to the following equation:

M=A(DE/T(2Cp−ECs)(Cs)t)^{1/2}

It will be noted in the above relationship that the amount of drug released is generally proportional to the square root of time. Assuming factors such as matrix porosity and tortuosity are constant within the dosage form, a plot of amount of drug released versus the square root of time should be linear.

It is often practical to design dosage forms which use a combination of the above-described mechanisms to achieve a particularly desirable release profile for a particular active ingredient. It will be readily recognized by those skilled in the art that a dosage form construct which offers multiple compartments, such as for example multiple core portions and/or multiple shell portions, is particularly advantageous for its flexibility in providing a number of different mechanisms for controlling the release of one or more active ingredients.

Various dosage forms have been proposed to approach a constant dissolution rate by employing dosage form shapes in which the surface area of contact between the drug and dissolution medium increase at the same rate as the path-length for diffusion. Most involve coating a portion of the dosage form with an impermeable layer to control the surface area available for dissolution of the drug. See for example, U.S. Pat. Nos. 3,146,169; 3,851,638; 4,663,147; 4,816,262; and 6,110,500. One shape of particular interest has been that of a torus. Another has been that of a truncated cone. The primary limitation of such designs has been laborious manufacturing processes which typically include making a core, coating the core with impermeable material, then removing a portion of the core and coating to create the area for drug dissolution. These types of processes have not been shown to be suitable for commercial scale manufacture.

Conventional modifed release systems may be prepared by compression, to produce either multiple stacked layers, or core and shell configurations. Modified release dosage forms prepared via compression are exemplified in U.S. Pat. Nos. 5,738,874 and 6,294,200, and WO 99/51209. It is possible, via compression-coating, to produce a 2-portion shell, which may function as a barrier, or release delaying coating, however compression-coated systems are limited by the shell thickness and shell composition. Gunsel et al., “Compression-coated and layer tablets” in Pharmaceutical Dosage Forms—Tablets, edited by H. A. Lieberman, L. Lachman, J. B. Schwartz (2nd ed., rev. and expanded. Marcel Dekker, Inc.) pp. 247-284, for example discloses the thickness of compression coated shells is typically between 800 and 1200 microns. Because of these limitations, compression-coated dosage forms are not optimal for providing certain types of modified release, such as for example diffusion-controlled release which is not preceded by a lag-time. U.S. Pat. No. 5,738,874, discloses a 3-layer pharmaceutical compressed tablet capable of liberating one or more drugs at different release rates, in which an immediate release dose of active may be contained in a compressed coating layer, and the compressed coating layer has a weight which is 230% to 250% of the weight of the core, and a sustained release dose of active ingredient is contained in the core. Alternatively the outer compressed coating layer may function via an erosion mechanism to delay release of an active ingredient contained in the core. U.S. Pat. No. 5,464,633, for example, discloses delayed-release dosage forms in which an extermal coating layer was applied by a compression coating process. The coating level ranged from 105 percent to 140 percent of the weight of the core in order to yield product with the desired time delayed profile.

The edible composition, core, shell and dosage form of this invention comprise about 25 to about 40 weight percent of at least one non-aqueous carrier material having a melting temperature of less than about 45 degrees C., and about 15 to about 60 weight percent of at least one thermoplastic material which has a melting temperature greater than about 50 degrees C. The edible composition, core, shell and dosage form of this invention may be prepared using “solvent-free” methods and methods using injection molding. As used herein, a “solvent-free” method refers to a method of making an edible composition, core, shell or dosage form in which the mass balance of components sums to zero: i.e. all components in the initial composition are present in the final composition.

In contrast, current core-shell systems are limited by the available methods for manufacturing them, as well as the materials that are suitable for use with the current methods. A shell, or coating, which confers modified release properties is typically applied via conventional methods, such as for example, spray-coating in a coating pan. Pan-coating produces a single shell which essentially surrounds the core. The single shell is inherently limited in its functionality. It is possible via pan-coating to apply multiple concentric shells, each with a different functionality, however such systems are limited in that the outer shell must first dissolve before the functionality conferred by each successive layer can be realized. It is also known, via pan coating, to deliver a first dose of active ingredient from a coating, and a second dose of active ingredient from a core. Dosage forms having sprayed coatings which provide delayed release are described, for example, in Maffione et al., “High-Viscosity HPMC as a Film-Coating Agent,” Drug Development and Industrial Pharmacy (1993) 19(16), pp. 2043-2053. U.S. Pat. No. 4,576,604, for example, discloses an osmotic device (dosage form) comprising a drug compartment surrounded by a wall (coating) in which the coating may comprise an immediate release dose of drug, and the inner drug compartment may comprise a sustained release dose of drug. The coating compositions that can be applied via spraying are limited by their viscosity. High viscosity solutions are difficult or impractical to pump and deliver through a spray nozzle. Spray coating methods suffer the further limitations of being time-intensive and costly. Several hours of spraying may be required to spray an effective amount of coating to control the release of an active ingredient. Coating times of 8 to 24 hours are not uncommon.

Alternately, conventional modified release systems may be prepared by compression, to produce either multiple stacked layers, or core and shell configurations. Modified release dosage forms prepared via compression are exemplified in U.S. Pat. Nos. 5,738,874 and 6,294,200, and WO 99/51209. It is possible, via compression-coating, to produce a 2-portion shell, which may function as a barrier, or release delaying coating, however compression-coated systems are limited by the shell thickness and shell composition. Gunsel et al., “Compression-coated and layer tablets” in Pharmaceutical Dosage Forms—Tablets, edited by H. A. Lieberman, L. Lachman, J. B. Schwartz (2nd ed., rev. and expanded. Marcel Dekker, Inc.) pp. 247-284, for example, discloses the thickness of compression coated shells is typically between 800 and 1200 microns. Because of these limitations, compression-coated dosage forms are not optimal for providing certain types of modified release, such as for example diffusion-controlled release which is not preceded by a lag-time. U.S. Pat. No. 5,738,874, discloses a 3-layer pharmaceutical compressed tablet capable of liberating one or more drugs at different release rates, in which an immediate release dose of active may be contained in a compressed coating layer, and the compressed coating layer has a weight which is 230% to 250% of the weight of the core, and a sustained release dose of active ingredient is contained in the core. Alternatively the outer compressed coating layer may function via an erosion mechanism to delay release of an active ingredient contained in the core. U.S. Pat. No. 5,464,633, for example, discloses delayed-release dosage forms in which an external coating layer was applied by a compression coating process. The coating level ranged from 105 percent to 140 percent of the weight of the core in order to yield product with the desired time delayed profile.

It is one object of this invention to provide an edible solid composition. It is another object of this invention to provide a core containing such an edible composition for use in a dosage form. It is yet another object of this invention to provide a shell containing such an edible composition for use in a dosage form. It is yet another object of this invention to provide a dosage form per se which contains such an edible composition. It is yet another object of this invention to provide a method for preparing the edible solid composition, core, shell or dosage form of this invention.

It is one feature of this invention that the edible solid composition contains at least one non-aqueous carrier material which has a melting temperature of less than about 45 degrees C. and at least one thermoplastic material which has a melting temperature greater than about 50 degrees C. It is another feature of this invention that the non-aqueous carrier material remains a part of the final edible solid composition. It is yet another feature of this invention that the non-aqueous carrier material enables pumping and flowability of high levels of meltable solids. It is yet another feature of this invention that the non-aqueous carrier material may plasticize the final edible solid composition.

It is one advantage of this invention that no water or organic solvents are required to prepare the edible solid composition of this invention, and thus no evaporation of solvent during drying is required. Accordingly, this invention is particularly useful in “solvent-free” methods of preparing edible solid compositions. It is another advantage of this invention that the edible solid composition of this invention may be employed in injection molding processes for preparing cores, shells, dosage forms and the like. It is yet another advantage of this invention that high concentrations of the non-aqueous carrier material may be incorporated into the final solid edible composition of this invention. Incorporation of non-aqueous carrier at these levels beneficially plasticizes the composition, facilitates removal from the mold, confers breakage resistance, improving the suitability of the composition for further processing, e.g. packaging operations, and eliminates the need for drying, providing economy in both energy utilization and throughtput. Other objects, features and advantages of this invention will be apparent to those skilled in the art from the detailed description set forth below.

### SUMMARY OF THE INVENTION

The present invention relates to an edible solid composition comprising: a) about 25 to about 40 weight percent based on the weight of the edible composition of at least one non-aqueous carrier material which has a melting temperature less than about 45 degrees C.; and b) about 15 to about 60 weight percent based on the weight of the edible composition of at least one thermoplastic material which has a melting temperature greater than about 50 degrees C.

The present invention also provides a dosage form comprising: (I) an edible solid composition comprising: a) about 25 to about 40 weight percent of at least one non-aqueous carrier material which has a melting temperature less than about 45 degrees C., and b) about 15 to about 60 weight percent of at least one thermoplastic material which has a melting temperature greater than about 50 degrees C.; and (II) at least one active ingredient.

The present invention further provides an edible solid composition prepared by a process comprising: a) admixing the following components: (i) about 25 to about 40 weight percent of at least one non-aqueous carrier material which has a melting temperature less than about 45 degrees C., and (ii) about 15 to about 60 weight percent of at least one thermoplastic material which has a melting temperature greater than about 50 degrees C.; b) providing the admixture into a mold at a temperature in the range of about 0 to about 40 degrees C.; c) heating the mold and admixture contained therein to a temperature in the range of about 50 to about 100 degrees C.; and d) cooling the mold and admixture contained therein to a temperature in the range of about 0 to about 25 degrees C.

The present invention also relates to a method for preparing an edible solid composition, wherein the method comprises: a) admixing the following components: (i) about 25 to about 40 weight percent of at least one non-aqueous carrier material which has a melting temperature less than about 45 degrees C., and (ii) about 15 to about 60 weight percent of at least one thermoplastic material which has a melting temperature greater than about 50 degrees C.; b) providing the admixture into a mold at a temperature in the range of about 0 to 40 degrees C.; c) heating the mold and admixture contained therein to a temperature in the range of about 50 to 100 degrees C.; and d) cooling the mold and admixture contained therein to a temperature in the range of about 0 to about 25 degrees C.

The present invention further relates to a modified release solid dosage form comprising one or more active ingredients, and an edible solid composition comprising: a) about 25 to about 40 weight percent of at least one non-aqueous carrier material which has a melting temperature less than about 45 degrees C.; and b) about 15 to about 60 weight percent of at least one thermoplastic material which has a melting temperature greater than about 50 degrees C.

### DETAILED DESCRIPTION OF THE INVENTION

The edible solid composition of this invention comprises: (a) about 25 to about 40 weight percent based upon the weight of the edible composition of at least one non-aqueous carrier material which has a melting temperature less than about 45 degrees C.; and (b) about 15 to about 60 weight percent based on hte weight of the edible composition of at least one thermoplastic material which has a melting temperature greater than about 50 degrees C.

In one embodiment, the non-aqueous carrier material is non-volatile.

In another embodiment, the non-aqueous carrier material has a melting point less than about 25 degrees C.

In another embodiment, the non-aqueous carrier material is at least one of mineral oil, propylene glycol, glycerin, polyethylene glycol having a molecular weight in the range of about 1000 to about 20,000, vegetable oil, castor oil, hydrogenated vegetable oils, palm kernel oil, cottonseed oil, sunflower oil, soybean oil, dibutyl sebacate, triethyl citrate, tributyl citrate, triacetin, diethyl phthalate, dibutyl phthalate, dimethyl phthalate, acetyltributyl citrate, acetyltriethyl citrate, polyoxyethylene alkyl ethers, polyethoxylated castor oil such as available under the tradename CREMOPHOR, polyoxyethylenesorbatan fatty acid esters such as those available under the tradename TWEEN and combinations thereof.

Surprisingly and advantageously, the final edible solid core, shell or dosage form of the invention is substantially solid, even though the non-aqueous carrier has been incorporated therein at a relatively high level. The non-aqueous carrier material may function to plasticize the final edible solid, core, shell or dosage form of this invention. One or more components (e.g. active ingredient) may be dispersed, e.g. dissolved or suspended in the non-aqueous carrier material.

In one embodiment, the thermoplastic material is at least one of polyvinyl acetate, polyalkylene glycols such as polyethylene glycol having a molecular weight in the range of about 1000 to about 20,000, or polyethylene oxide; shellac, polycapractones, polyvinyl alcohol, cetyl alcohol, or combinations thereof.

In another embodiment, the edible solid composition additionally comprises up to about 40 weight percent based on the weight of the solid composition of at least one compatibility material for retaining the non-aqueous carrier material in the edible solid composition, and preventing the carrier material from separating or leaching upon cooling. Without wishing to be bound by any one theory, it is believed that the compatibility material aids in enabling the non-aqueous carrier material to be dispersed in and remain a part of the final solid edible composition or dosage form. The compatibility material may be at least one of emulsifiers, acrylic polymers, cellulosic polymers, waxes or combinations thereof.

In one embodiment, the compatibility material comprises a cellulosic polymer selected from the group consisting of sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, hydroxypropylethylcellulose, ethylcellulose, cellulose acetate and its derivatives, and derivatives and combinations thereof.

In one embodiment, the compatibility material is a wax selected from the group consisting of camauba wax, spermaceti wax, beeswax, candelilla wax, shellac wax, microcrystalline wax, and paraffin wax or combinations thereof.

In another embodiment, the compatibility material is a fatty acid ester, an anionic methacrylic polymer, or combinations thereof. Suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GLYCOWAX-932, lauroyl macrogol-32 glycerides, and stearoyl macrogol-32 glyceride;.

In one embodiment, the fatty acid ester is at least one of glyceryl monostearate, glyceryl palmitostearate, glyceryl behenate, or combinations thereof.

In one embodiment, the anionic methacrylic polymer is an anionic methacrylic copolymer having less than about 35% methacrylic acid units on a molar basis (such as those available from Rohm Pharma GmbH from under the tradename EUDRAGIT S100).

In another embodiment, the compatibility material may function as a release-modifying excipient, to provide a modification to the release of one or more active ingredients contained in either the composition of the invention, or an underlying core portion of the dosage form.

The edible solid composition of this invention may be employed in cores or shells for use in a dosage form, or dosage forms per se which contain or are prepared from such an edible solid composition. The edible solid composition of this invention is particularly useful in “solvent-free” methods of preparing cores, shells or dosage forms and in methods for preparing cores, shells or dosage forms using injection molding.

The edible solid composition of this invention is also useful for providing a diffusional matrix, a diffusional membrane, or an impermeable barrier. In one embodiment, the edible solid composition is employed as a diffusional matrix in a core, core portion, or dosage form per se. In this embodiment, the release of one or more active ingredients dispersed throughtout the edible solid composition are modified (e.g. controlled, sustained, prolonged, extended, and the like). In another embodiment, the edible solid composition is employed as a diffusional membrane in a shell or shell portion of a dosage form. In this embodiment, the release of one or more active ingredients contained in an underlying portion of the dosage form are modified (e.g. controlled, sustained, prolonged, extended, and the like). In yet another embodiment, the edible solid composition is employed as an impermeable barrier, for example, covering a portion of the surface of a dosage form. In one such embodiment, the edible composition functions to limit the surface area available for release of active ingredient from the dosage form. In another embodiment in which the edible solid composition functions as an impermeable barrier, the edible solid composition is located between first and second portions of a dosage form, for example for the purpose of preventing passage therethrough of active ingredient or ingredients from the first or second portion of the dosage form.

The edible solid composition of the present invention may be formulated to be impermeable or diffusable, and may be incorporated into the core or a core portion or shell or a shell portion of a modified release dosage form, or may be employed as a dosage form per se.

As used herein, the term “dosage form” applies to any solid, semi-solid, or liquid composition designed to contain a specific pre-determined amount (dose) of a certain ingredient, for example an active ingredient as defined below. Suitable dosage forms may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. Preferably the dosage forms of the present invention are considered to be solid, however they may contain liquid or semi-solid components. In a particularly preferred embodiment, the dosage form is an orally administered system for delivering a pharmaceutical active ingredient to the GI tract of a human.

The dosage forms of this invention exhibit modified release of one or more active ingredients contained therein. The active ingredient or ingredients may be found within the core, the shell, or a portion or combination thereof. As used herein, the term “modified release” shall apply to dosage forms, coatings, shells, cores, portions thereof, or compositions that alter the release of an active ingredient in any manner. The active ingredient or ingredients that are released in a modified manner may be contained within the coating, shell, core, composition, or portion thereof providing the modification. Alternatively the modified release active ingredient may be contained in a different portion of the dosage form from the coating, shell, core, composition, or portion thereof providing the modification; for example the modified release active ingredient may be contained in a core portion, and the modification may be provided by the overlaying shell portion. Types of modified release include controlled, prolonged, sustained, extended, delayed, pulsatile, repeat action, and the like. Suitable mechanisms for achieving these types of modified release include diffusion, erosion, surface area control via geometry and/or impermeable barriers, or other mechanisms known in the art. Moreover, the modified release properties of the dosage form may be achieved through design of the core or a portion thereof, or the shell or portion thereof, or a combination of two or more of these parts of the dosage form.

The dosage forms of this invention are designed to release substantially all (i.e. at least about 80%, or at least about 90%, say at least about 95%) of the active ingredient contained therein, within a specified amount of time. As used herein, the total amount of time required for substantially all of the active ingredient or ingredients to be released from the dosage form shall be referred to as the “dosing interval.” During the dosing interval, the amount of drug released is typically measured at several time points.

As used herein, the “release rate” of an active ingredient (e.g., drug) refers to the quantity of active ingredient released from a dosage form per unit time, e.g., milligrams of active ingredient released per hour (mg/hr). Active ingredient rates are calculated under in vitro dosage form dissolution testing conditions known in the art. As used herein, an active ingredient rate obtained at a specified time “following administration” refers to the in vitro active ingredient release rate obtained at the specified time following implementation of an appropriate dissolution test.

As used herein, a “constant release rate” is obtained over a given time interval when the periodic release rates determined during two or more portions of the time interval are substantially the same, i.e. not more than 6% different. As used herein, “non-constant release rate” shall mean two or more periodic release rates are not the same, i.e. more than 6% different, over the entire duration of the specified interval.

Suitable active ingredients for use in this invention include for example pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, GI agents, migraine preparations, motion sickness products, mucolytics, muscle relaxants, osteoporosis preparations, polydimethylsiloxanes, respiratory agents, sleep-aids, urinary tract agents and mixtures thereof.

Suitable oral care agents include breath fresheners, tooth whiteners, antimicrobial agents, tooth mineralizers, tooth decay inhibitors, topical anesthetics, mucoprotectants, and the like.

Suitable flavorants include menthol, peppermint, mint flavors, fruit flavors, chocolate, vanilla, bubblegum flavors, coffee flavors, liqueur flavors and combinations and the like.

Examples of suitable GI agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotadine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In one embodiment of the invention, the active ingredient may be selected from bisacodyl, famotadine, ranitidine, cimetidine, prucalopride, diphenoxylate, loperamide, lactase, mesalamine, bismuth, antacids, and pharmaceutically acceptable salts, esters, isomers, and mixtures thereof.

In another embodiment, the active ingredient is selected from analgesics, anti-inflammatories, and antipyretics, e.g. non-steroidal anti-inflammatory drugs (NSAIDs), including propionic acid derivatives, e.g. ibuprofen, naproxen, ketoprofen and the like; acetic acid derivatives, e.g. indomethacin, diclofenac, sulindac, tolmetin, and the like; fenamic acid derivatives, e.g. mefenamic acid, meclofenamic acid, flufenamic acid, and the like; biphenylcarbodylic acid derivatives, e.g. diflunisal, flufenisal, and the like; and oxicams, e.g. piroxicam, sudoxicam, isoxicam, meloxicam, and the like. In one embodiment, the active ingredient is selected from propionic acid derivative NSAID, e.g. ibuprofen, naproxen, flurbiprofen, fenbufen, fenoprofen, indoprofen, ketoprofen, fluprofen, pirprofen, carprofen, oxaprozin, pranoprofen, suprofen, and pharmaceutically acceptable salts, derivatives, and combinations thereof. In another embodiment of the invention, the active ingredient may be selected from acetaminophen, acetyl salicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof.

In another embodiment of the invention, the active ingredient may be selected from pseudoephedrine, phenylpropanolamine, chlorpheniramine, dextromethorphan, diphenhydramine, astemizole, terfenadine, fexofenadine, loratadine, desloratadine, cetirizine, mixtures thereof and pharmaceutically acceptable salts, esters, isomers, and mixtures thereof.

Examples of suitable polydimethylsiloxanes, which include, but are not limited to dimethicone and simethicone, are those disclosed in U.S. Pat. Nos. 4,906,478, 5,275,822, and 6,103,260, the contents of each is expressly incorporated herein by reference. As used herein, the term “simethicone” refers to the broader class of polydimethylsiloxanes, including but not limited to simethicone and dimethicone.

The active ingredient is present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. Typically, the dosage fonn comprises at least about 1 weight percent, preferably, the dosage form comprises at least about 5 weight percent, e.g. about 20 weight percent of a combination of one or more active ingredients.

The active ingredient may be present in the dosage form in any form. For example, the active ingredient may be dispersed at the molecular level, e.g. melted or dissolved, within the dosage form, or may be in the form of particles, which in turn may be coated or uncoated. If the active ingredient is in form of particles, the particles (whether coated or uncoated) typically have an average particle size of about 1-2000 microns. In one preferred embodiment, such particles are crystals having an average particle size of about 1-300 microns. In another embodiment, the particles are granules or pellets having an average particle size of about 50-2000 microns, preferably about 50-1000 microns, most preferably about 100-800 microns.

At least a portion of the active ingredient may be optionally coated with a release-modifying coating, as known in the art. This advantageously provides an additional tool for modifying the release profile of active ingredient from the dosage form. For example, the core may contain coated particles of one or more active ingredients, in which the particle coating confers a release modifying function, as is well known in the art. Examples of suitable release modifying coatings for particles are described in U.S. Pat. Nos. 4,173,626; 4,863,742; 4,980,170; 4,984,240; 5,286,497; 5,912,013; 6,270,805; and 6,322,819. Commercially available modified release coated active particles may also be employed. Accordingly, all or a portion of one or more active ingredients in the core may be coated with a release-modifying material.

The active ingredient or ingredients may be located in any portion of the dosage form, for example in a core, a first coating layer, a shell, an outer coating layer, or any portion thereof.

In embodiments in which it is desired for the active ingredient to be absorbed into the systemic circulation of an animal, the active ingredient or ingredients are preferably capable of dissolution upon contact with a fluid such as water, gastric fluid, intestinal fluid or the like.

In one embodiment, the dissolution characteristics of one or more active ingredients are modified: e.g. controlled, sustained, extended, retarded, prolonged, delayed and the like by the edible solid composition of the invention. The active ingredients having the modified release characteristics may be dispersed throughout the edible solid composition, or may be contained in an underlying portion of the dosage form. In one embodiment in which one or more active ingredients are released in a modified manner, the modified release active ingredient or ingredients are contained in the core. In one particular such embodiment, the dosage form releases one or more active ingredients contained in the core at a substantially constant rate over a specified time interval.

In certain optional embodiments, in which the edible composition of the invention is incorporated into a dosage form which is further designed to deliver an immediate release dose of one or more active ingredients, the dissolution characteristics of at least one active ingredient contained in the core meets USP specifications for immediate release tablets containing the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19 -20 and 856 (1999).

In certain other embodiments, the edible solid composition of the invention is employed in a core or core portion which functions as a diffusional matrix. In these embodiments, the core or core portion comprises active ingredient distributed throughout the edible solid composition. The edible solid composition has the form of an insoluble porous matrix, which contains pores or channels through which fluids can enter the core, and the active ingredient must diffuse to be released from the dosage form. In these embodiments, the rate of active ingredient release from the core or core portion will depend upon the area (A) of the matrix, the diffusion coefficient (D), the porosity (E) and tortuosity (T) of the matrix, the drug solubility (Cs) in the dissolution medium, and the drug concentration (Cp) in the dosage form. In embodiments in which the core or core portion functions as a diffusional matrix, the release of the active ingredient from the core or core portion may be described as controlled, prolonged, sustained, or extended. In these embodiments, the contribution to active ingredient dissolution from the core or core portion may follow zero-order, first-order, or preferably square-root of time kinetics. In certain such embodiments, the non-aqueous carrier, or the thermoplastic material, or the optional compatability material may function as a pore former in the diffusional matrix core or core portion.

In certain other embodiments, the edible solid composition of the invention is employed in a core or core portion which functions as an erosional matrix In certain other embodiments, the edible solid composition of the invention is employed in a core or core portion which functions to relase active ingredient therefrom essentially immediately upon contact of the core or core portion with a suitable liquid medium. For example the core or core portion may be a component of a pulsatile release dosage form from which a portion, or dose of active ingredient is released essentially immediately following a programmed time delay caused by the erosion of a coating or shell portion on the surface thereof.

In embodiments in which the core or core portion functions to modify release of an active ingredient contained therein, the release of active ingredient may be further modified by the function of a shell surrounding the core, or a shell portion residing upon at least a portion of the core or core portion. In such embodiments, the release of the active ingredient from the dosage form will be governed by the sum of all the contributions acting upon it, e.g. from the relevant core or core portion and shell or shell portion, and may be described as controlled, prolonged, sustained, extended, delayed, or pulsatile. In these embodiments, the dissolution of active ingredient from the dosage form may follow zero-order, first-order, or square-root of time kinetics.

The core may be in a variety of different shapes. For example, the core may be shaped as a polyhedron, such as a cube, pyramid, prism, or the like; or may have the geometry of a space figure with some non-flat faces, such as a cone, truncated cone, cylinder, sphere, torus, or the like. In certain embodiments, the core has one or more major faces. For example in embodiments wherein the core is a compressed tablet, the core surface typically has two opposing major faces formed by contact with the upper and lower punch faces in the compression machine. In such embodiments the core surface typically further comprises a “belly-band” located between the two major faces, and formed by contact with the die walls in the compression machine. Exemplary core shapes which may be employed include tablet shapes formed from compression tooling shapes described by “The Elizabeth Companies Tablet Design Training Manual” (Elizabeth Carbide Die Co., Inc., p. 7 (McKeesport, Pa.) (incorporated herein by reference) as follows (the tablet shape corresponds inversely to the shape of the compression tooling):

Shallow Concave.

Standard Concave.

Deep Concave.

Extra Deep Concave.

Modified Ball Concave.

Standard Concave Bisect.

Standard Concave Double Bisect.

Standard Concave European Bisect.

Standard Concave Partial Bisect.

Double Radius.

Bevel & Concave.

Flat Plain.

Flat-Faced-Beveled Edge (F.F.B.E.).

F.F.B.E. Bisect.

F.F.B.E. Double Bisect.

Ring.

Dimple.

Ellipse.

Oval.

Capsule.

Rectangle.

Square.

Triangle.

Hexagon.

Pentagon.

Octagon.

Diamond.

Arrowhead.

Bullet.

Shallow Concave.

Standard Concave.

Deep Concave.

Extra Deep Concave.

Modified Ball Concave.

Standard Concave Bisect.

Standard Concave Double Bisect.

Standard Concave European Bisect.

Standard Concave Partial Bisect.

Double Radius.

Bevel & Concave.

Flat Plain.

Flat-Faced-Beveled Edge (F.F.B.E.).

F.F.B.E. Bisect.

F.F.B.E. Double Bisect.

Ring.

Dimple.

Ellipse.

Oval.

Capsule.

Rectangle.

Square.

Triangle.

Hexagon.

Pentagon.

Octagon.

Diamond.

Arrowhead.

Bullet.

Barrel.

Half Moon.

Shield.

Heart.

Almond.

House/Home Plate.

Parallelogram.

Trapezoid.

FIG. 8/Bar Bell.

Bow Tie.

Uneven Triangle.

In one embodiment of the invention, the core comprises multiple portions, for example a first portion and a second portion. The portions may be prepared by the same or different methods and mated using various techniques, such as the thermal cycle molding molding methods described herein. For example, the first and second portions may both be made by compression, or both may be made by molding. Or one portion may be made by compression and the other by molding. The same or different active ingredient may be present in the first and second portions of the core. Alternately, one or more core portions may be substantially free of active ingredients.

In certain other embodiments, the core comprises multiple portions which comprise different active ingredients or have different release-modifying properties, or both; and the shell comprises a corresponding number of multiple portions, which each cover a specific core portion to modify or further modify the release of one or more active ingredients contained within the respective core portion. For such embodiments, it is critical to have a manufacturing process which is capable of maintaining the orientation of the core prior to and during the application of the shell or each shell portion thereon. Advantageously, the orientation of the components of the dosage forms of the present invention can be precisely controlled, when manufactured using the thermal cycle apparatus and described below. In one such embodiment, the dosage form comprises a core comprising a first core portion and a second core portion which are compositionally different, wherein at least one of the first or second core portions comprises an active ingredient; and a shell which surrounds the core and comprises a first shell portion and a second shell portion which are compositionally different, wherein at least one of the first or second shell portions confers a modification to the release of an active ingredient contained in the underlying core portion.

In certain embodiments, the core or dosage form may further comprise a water-impermeable barrier layer between first and second core portions. The water-impermeable barrier layer may be made by any method, for example compression or molding, and preferably comprises at least one water-insoluble material selected from water-insoluble polymers, insoluble edible materials, pH-dependent polymers, and mixtures thereof.

The core or core portion of the present invention may be prepared by any suitable method, including for example compression and molding, and depending on the method by which it is made, typically comprises active ingredient and a variety of excipients (inactive ingredients which may be useful for conferring desired physical properties to the core or core portion).

In embodiments in which the core, or a portion thereof, is made by compression, suitable excipients include fillers, binders, disintegrants, lubricants, glidants, and the like, as known in the art. In embodiments in which the core or core portion is made by compression and additionally confers modified release of an active ingredient contained therein, the core or core portion preferably further comprises a release-modifying compressible excipient.

[Yes]

Suitable fillers for use in making the core, or a portion thereof, by compression include water-soluble compressible carbohydrates such as sugars, which include dextrose, sucrose, maltose, and lactose, sugar-alcohols, which include mannitol, sorbitol, maltitol, xylitol, starch hydrolysates, which include dextrins, and maltodextrins, and the like, water insoluble plastically deforming materials such as microcrystalline cellulose or other cellulosic derivatives, water-insoluble brittle fracture materials such as dicalcium phosphate, tricalcium phosphate and the like and mixtures thereof.

Suitable binders for making the core, or a portion thereof, by compression include dry binders such as polyvinyl pyrrolidone, hydroxypropylmethylcellulose, and the like; wet binders such as water-soluble polymers, including hydrocolloids such as acacia, alginates, agar, guar gum, locust bean, carrageenan, carboxymethylcellulose, tara, gum arabic, tragacanth, pectin, xanthan, gellan, gelatin, maltodextrin, galactomannan, pusstulan, laminarin, scleroglucan, inulin, whelan, rhamsan, zooglan, methylan, chitin, cyclodextrin, chitosan, polyvinyl pyrrolidone, cellulosics, sucrose, starches, and the like; and derivatives and mixtures thereof.

Suitable disintegrants for making the core, or a portion thereof, by compression, include sodium starch glycolate, cross-linked polyvinylpyrrolidone, cross-linked carboxymethylcellulose, starches, microcrystalline cellulose, effervescent compounds, effervescent mixtures, and the like, and combinations thereof. As used herein, “effervescent” is meant to include inorganic salts of carbonic acid, inorganic bicarbonate salts, acid/base pairs that react to liberate gases, and the like.

Suitable lubricants for making the core, or a portion thereof, by compression include long chain fatty acids and their salts, such as magnesium stearate and stearic acid, talc, glycerides and waxes.

Suitable glidants for making the core, or a portion thereof, by compression, include colloidal silicon dioxide, and the like.

Suitable release-modifying compressible excipients for making the core, or a portion thereof, by compression include swellable erodible hydrophillic materials, insoluble edible materials, pH-dependent polymers, and the like.

Suitable swellable erodible hydrophilic materials for use as release-modifying excipients for making the core, or a portion thereof, by compression include: water swellable cellulose derivatives, polyalkalene glycols, thermoplastic polyalkalene oxides, acrylic polymers, hydrocolloids, clays, gelling starches, and swelling cross-linked polymers, and derivatives, copolymers, and combinations thereof. Examples of suitable water swellable cellulose derivatives include sodium carboxymethylcellulose, cross-linked hydroxypropylcellulose, hydroxypropyl cellulose (HPC), hydroxypropylmethylcellulose (HPMC), hydroxyisopropylcellulose, hydroxybutylcellulose, hydroxyphenylcellulose, hydroxyethylcellulose (HEC), hydroxypentylcellulose, hydroxypropylethylcellulose, hydroxypropylbutylcellulose, hydroxypropylethylcellulose. Examples of suitable polyalkalene glycols include polyethylene glycol. Examples of suitable thermoplastic polyalkalene oxides include poly (ethylene oxide). Examples of suitable acrylic polymers include potassium methacrylatedivinylbenzene copolymer, polymethylmethacrylate, CARBOPOL (high-molecular weight cross-linked acrylic acid homopolymers and copolymers), and the like. Examples of suitable hydrocolloids include alginates, agar, guar gum, locust bean gum, kappa carrageenan, iota carrageenan, tara, gum arabic, tragacanth, pectin, xanthan gum, gellan gum, maltodextrin, galactomannan, pusstulan, laminarin, scleroglucan, gum arabic, inulin, pectin, gelatin, whelan, rhamsan, zooglan, methylan, chitin, cyclodextrin, chitosan. Examples of suitable clays include smectites such as bentonite, kaolin, and laponite; magnesium trisilicate, magnesium aluminum silicate, and the like, and derivatives and mixtures thereof. Examples of suitable gelling starches include acid hydrolyzed starches, swelling starches such as sodium starch glycolate, and derivatives thereof. Examples of suitable swelling cross-linked polymers include cross-linked polyvinyl pyrrolidone, cross-linked agar, and cross-linked carboxymethylcellose sodium.

Suitable insoluble edible materials for use as release-modifying excipients for making the core, or a portion thereof, by compression include water-insoluble polymers, and low-melting hydrophobic materials. Examples of suitable water-insoluble polymers include ethylcellulose, polyvinyl alcohols, polyvinyl acetate, polycaprolactones, cellulose acetate and its derivatives, acrylates, methacrylates, acrylic acid copolymers; and the like and derivatives, copolymers, and combinations thereof. Suitable low-melting hydrophobic materials include fats, fatty acid esters, phospholipids, and waxes. Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GLYCOWAX-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes include camauba wax, spermaceti wax, beeswax, candelilla wax, shellac wax, microcrystalline wax, and paraffin wax; fat-containing mixtures such as chocolate; and the like.

Suitable pH-dependent polymers for use as release-modifying excipients for making the core, or a portion thereof, by compression include enteric cellulose derivatives, for example hydroxypropyl methylcellulose phthalate, hydroxypropyl methylcellulose acetate succinate, cellulose acetate phthalate; natural resins such as shellac and zein; enteric acetate derivatives such as for example polyvinylacetate phthalate, cellulose acetate phthalate, acetaldehyde dimethylcellulose acetate; and enteric acrylate derivatives such as for example polymethacrylate-based polymers such as poly(methacrylic acid, methyl methacrylate) 1:2, which is commercially available from Rohm Pharma GmbH under the tradename EUDRAGIT S, and poly(methacrylic acid, methyl methacrylate) 1:1, which is commercially available from Rohm Pharma GmbH under the tradename EUDRAGIT L, and the like, and derivatives, salts, copolymers, and combinations thereof.

Suitable pharmaceutically acceptable adjuvants for making the core, or a portion thereof, by compression include, preservatives; high intensity sweeteners such as aspartame, acesulfame potassium, sucralose, and saccharin; flavorants; colorants; antioxidants; surfactants; wetting agents; and the like and mixtures thereof.

In embodiments in which the core or a portion thereof is prepared by compression, a dry blending (i.e. direct compression), or wet granulation process may be employed. In a dry blending (direct compression) method, the active ingredient or ingredients, together with the excipients, are blended in a suitable blender, than transferred directly to a compression machine for pressing into tablets. In a wet granulation method, the active ingredient or ingredients, appropriate excipients, and a solution or dispersion of a wet binder (e.g. an aqueous cooked starch paste, or solution of polyvinyl pyrrolidone) are mixed and granulated. Alternatively a dry binder may be included among the excipients, and the mixture may be granulated with water or other suitable solvent. Suitable equipment for wet granulation are known in the art, including low shear, e.g. planetary mixers; high shear mixers; and fluid beds, including rotary fluid beds. The resulting granulated material is dried, and optionally dry-blended with further ingredients, e.g. adjuvants and/or excipients such as for example lubricants, colorants, and the like. The final dry blend is then suitable for compression. Methods for direct compression and wet granulation processes are known in the art, and are described in detail in, for example, Lachman, et al., The Theory and Practice of Industrial Pharmacy, Chapter 11 (3rd ed. 1986).

The dry-blended, or wet granulated, powder mixture is typically compacted into tablets using a rotary compression machine as known in the art, such as for example those commercially available from Fette America Inc. (Rockaway, N.J.), or Manesty Machines LTD (Liverpool, UK). In a rotary compression machine, a metered volume of powder is filled into a die cavity, which rotates as part of a “die table” from the filling position to a compaction position where the powder is compacted between an upper and a lower punch to an ejection position, where the resulting tablet is pushed from the die cavity by the lower punch and guided to an ejection chute by a stationary “take-off” bar.

In one particular embodiment, the core or core portion may be prepared by the compression methods and apparatus described in copending U.S. patent application Ser. No. 09/966,509, pages 16-27, the disclosure of which is incorporated herein by reference. Specifically, the core is made using a rotary compression module comprising a fill zone, insertion zone, compression zone, ejection zone, and purge zone in a single apparatus having a double row die construction as shown in FIG. 6 of U.S. patent application Ser. No. 09/966,509. The dies of the compression module are preferably filled using the assistance of a vacuum, with filters located in or near each die. The purge zone of the compression module includes an optional powder recovery system to recover excess powder from the filters and return the powder to the dies.

In certain preferred embodiments of this invention, the dosage form, core, or the shell, or a portion thereof, is prepared by molding. In such embodiments, the dosage form, core, or the shell, or a portion thereof, is made from a flowable material which contains the edible solid composition of this invention.

The flowable material may optionally comprise adjuvants or excipients, which may comprise up to about 30% by weight of the flowable material. Examples of suitable adjuvants or excipients include plasticizers, detackifiers, humectants, surfactants, anti-foaming agents, colorants, flavorants, sweeteners, opacifiers, and the like. Suitable plasticizers for making the core, the shell, or a portion thereof, by molding include, but not be limited to polyethylene glycol; propylene glycol; glycerin; sorbitol; triethyl citrate; tribuyl citrate; dibutyl sebecate; vegetable oils such as castor oil, rape oil, olive oil, and sesame oil; surfactants such as polysorbates, sodium lauryl sulfates, and dioctyl-sodium sulfosuccinates; mono acetate of glycerol; diacetate of glycerol; triacetate of glycerol; natural gums; triacetin; acetyltributyl citrate; diethyloxalate; diethylmalate; diethyl fumarate; diethylmalonate; dioctylphthalate; dibutylsuccinate; glyceroltributyrate; hydrogenated castor oil; fatty acids; substituted triglycerides and glycerides; and the like and/or mixtures thereof. In one embodiment, the plasticizer is triethyl citrate. In certain embodiments, the shell is substantially free of plasticizers, i.e. contains less than about 1%, say less than about 0.01% of plasticizers.

In one embodiment, the flowable material comprises less than 5% humectants, or alternately is substantially free of humectants, such as glycerin, sorbitol, maltitol, xylitol, or propylene glycol. Humectants have traditionally been included in pre-formed films employed in enrobing processes, such as that disclosed in U.S. Pat. Nos. 5,146,730 and 5,459,983, to ensure adequate flexibility or plasticity and bondability of the film during processing. Humectants function by binding water and retaining it in the film. Pre-formed films used in enrobing processes can typically comprise up to 45% water. Disadvantageously, the presence of humectant prolongs the drying process, and can adversely affect the stability of the finished dosage form.

The core, the shell, or dosage form of this invention is molded using a solvent-free process, which is discussed further herein. In certain embodiments, the dosage form, core, shell, or portions thereof may comprise active ingredient contained within an excipient matrix. In certain other embodiments the core, shell or portions thereof comprising the composition of the present invention may be substantially free of active ingredient. The solvent-free process may be used to obtain semipermeable, impermeable, or diffusible shells or shell portions.

In one embodiment, the shell or shell portion_of this invention is made using a flowable material comprising the edible solid composition of this invention.

In certain embodiments, the shell or shell portion functions to slow or delay the rate of passage of a fluid, such as water or a biological fluid therethrough.

The shell or shell portion may advantageously be applied to a core directly by a molding process, yielding a uniform and homogeneous layer in 5 minutes or less, e.g. 60 seconds or less, or 30 seconds or less, or 10 seconds or less, and in certain embodiments, say 1 second or less.

A preferred method for making the shell or shell portion of this invention comprises: (a) preparing a dispersion of the non-aqueous carrier, thermoplastic material, optional compatability material, and other shell materials; (b) injecting a flowable shell material (the flowable shell material may be heated in a heated feed tank) into a mold cavity (at room temp or below) containing the core such that the flowable shell material surrounds a first portion of the core within the mold cavity; (c) rapidly cycling the temperature of the mold cavity from hot (e.g. about 70 to about 95° C.) to cold (e.g. about 0 to about 10° C.) to induce thermal setting of the flowable shell material surrounding the first portion of the core; (d) opening the mold cavity and rotating the portion of the mold containing the core to expose a second portion of the core; (e) closing the mold cavity; (f) injecting room temperature flowable shell material into the mold cavity such that the flowable shell material surrounds the second portion of the core within the mold cavity; (g) rapidly cycling the temperature of the mold cavity from hot (e.g. about 70 to about 95° C.) to cold (e.g. about 0 to about 10° C.) to induce thermal setting of the flowable shell material surrounding the second portion of the core; (h) removing the coated core from the mold cavity

The shell of the present invention has a cross-sectional area in the range of about 1 to 900 sq. mm, preferably about 25 to 400 sq. mm, most preferably about 50 to about 200 sq. mm.

In certain other embodiments of this invention, at least a portion of the shell functions as a diffusional membrane which contains pores through which liquid medium containing active ingredient within the dosage form can be released through the diffusible shell portion in a sustained, extended, prolonged or retarded manner. In these embodiments, the rate of release of active ingredient from the underlying core will depend upon the total pore area in the shell or shell portion, the pathlength of the pores, and the solubility and diffusivity of the active ingredient (in addition to its rate of release from the core or core portion itself). In preferred embodiments in which the shell or shell portion functions as a diffusional membrane, the release of the active ingredient from the dosage form may be described as controlled, prolonged, sustained or extended. In these embodiments, the contribution to active ingredient dissolution from the shell or shell portion may follow zero-order, first-order, or square-root of time kinetics. In certain such embodiments, the diffusional membrane shell or shell portion preferably comprises a release-modifying excipient such as a combination of a pore former and an insoluble edible material such as for example a film forming water insoluble polymer.

In embodiments in which the shell or portion thereof functions to modify the release of an active ingredient which is contained in the core or the subject shell or shell portion, the thickness of the shell or shell portion is critical to the release properties of the dosage form. Advantageously the dosage forms of the invention can be made with precise control over shell thickness. In a preferred embodiment in which the shell or one or more shell portions function to modify the release of an active ingredient which is contained in the core or the subject shell or shell portion, the shell or shell portion is made by the thermal cycle injection molding methods and apparatus described below.

In certain other embodiments, one or more shell portions function as a barrier to prevent release therethrough of an active ingredient contained in the underlying core or core portion. In such embodiments, active ingredient is typically released from a portion of the dosage form which is not covered by the barrier shell portion. Such embodiments advantageously allow for further control of the surface area for release of the active ingredient. In certain such embodiments, the barrier shell portion preferably comprises a water insoluble material such as for example a water insoluble polymer.

In certain other embodiments of the invention, a further degree of flexibility in designing the dosage forms of the present invention can be achieved through the use of an additional outer coating overlaying the shell or one or more portions thereof. The additional outer coating may be applied for example by compression, or by molding. In such embodiments, the dosage form of the invention comprises at least one active ingredient; a core; a shell or shell portion which resides upon at least a portion of the core; and an outer coating which covers at least a portion of the shell or shell portion. The outer coating may for example cover a portion of the first shell portion, or the second shell portion, or both, or may surround the entire shell. In one particularly preferred embodiment, the outer coating comprises an active ingredient, which is released immediately (i.e. the dissolution of the active ingredient from the outer coating conforms to USP specifications for immediate release dosage forms of the particular active ingredient employed). In one such particularly preferred embodiment, the dosage form is a pulsatile drug delivery system, in which one or more shell portions provides for delayed release of a second dose of active ingredient, which is contained in an underlying core portion.

In one embodiment of this invention, the shell or shell portion is substantially free of pores having a diameter of 0.5-5.0 microns. As used herein, “substantially free” means that the shell or shell portion has a pore volume of less than about 0.02 cc/g, preferably less than about 0.01 cc/g, more preferably less than about 0.005 cc/g in the pore diameter range of 0.5 to 5.0 microns. In contrast, typical compressed materials have pore volumes of more than about 0.02 cc/g in this diameter range. In another embodiment of this invention, the core is a molded core and the core or core portions are substantially free of pores having a diameter of 0.5-5.0 microns.

The pore volume, pore diameter and density of the shell or shell portion may be determined using a Quantachrome Instruments PoreMaster 60 mercury intrusion porosimeter and associated computer software program known as “Porowin.” The procedure is documented in the Quantachrome Instruments PoreMaster Operation Manual. The PoreMaster determines both pore volume and pore diameter of a solid or powder by forced intrusion of a non-wetting liquid (mercury), which involves evacuation of the sample in a sample cell (penetrometer), filling the cell with mercury to surround the sample with mercury, applying pressure to the sample cell by: (i) compressed air (up to 50 psi maximum); and (ii) a hydraulic (oil) pressure generator (up to 60000 psi maximum). Intruded volume is measured by a change in the capacitance as mercury moves from outside the sample into its pores under applied pressure. The corresponding pore size diameter (d) at which the intrusion takes place is calculated directly from the so-called “Washburn Equation”: d=−(4γ(cos θ))/P where γ is the surface tension of liquid mercury, θ is the contact angle between mercury and the sample surface and P is the applied pressure.

Equipment used for pore volume measurements:

Quantachrome Instruments PoreMaster 60.

Analytical Balance capable of weighing to 0.0001 g.

Desiccator.

Reagents used for measurements:

High purity nitrogen.

Triply distilled mercury.

High pressure fluid (Dila AX, available from Shell Chemical Co.).

Liquid nitrogen (for Hg vapor cold trap).

Isopropanol or methanol for cleaning sample cells.

Liquid detergent for cell cleaning.

Procedure: the samples remain in sealed packages or as received in the dessicator until analysis. The vacuum pump is switched on, the mercury vapor cold trap is filled with liquid nitrogen, the compressed gas supply is regulated at 55 psi., and the instrument is turned on and allowed a warm up time of at least 30 minutes. The empty penetrometer cell is assembled as described in the instrument manual and its weight is recorded. The cell is installed in the low pressure station and “evacuation and fill only” is selected from the analysis menu, and the following settings are employed:

Fine Evacuation time: 1 min.

Fine Evacuation rate: 10

Coarse Evacuation time: 5 min.

The cell (filled with mercury) is then removed and weighed. The cell is then emptied into the mercury reservoir, and two tablets from each sample are placed in the cell and the cell is reassembled. The weight of the cell and sample are then recorded. The cell is then installed in the low-pressure station, the low-pressure option is selected from the menu, and the following parameters are set:

Mode: Low pressure

Fine evacuation rate: 10

Fine evacuation until: 200 μ Hg

Coarse evacuation time: 10 min.

Fill pressure: Contact ±0.1

Maximum pressure: 50

Direction: Intrusion And Extrusion

Repeat: 0

Mercury contact angle: 140

Mercury surface tension: 480

Data acquisition is then begun. The pressure vs. cumulative volume-intruded plot is displayed on the screen. After low-pressure analysis is complete, the cell is removed from the low-pressure station and reweighed. The space above the mercury is filled with hydraulic oil, and the cell is assembled and installed in the high-pressure cavity. The following settings are used:

Mode: Fixed rate

Motor speed: 5

Start pressure: 20

End pressure: 60,000

Direction: Intrusion and extrusion

Repeat: 0

Oil fill length: 5

Mercury contact angle: 140

Mercury surface tension: 480

Data acquisition is then begun and graphic plot pressure vs. intruded volume is displayed on the screen. After the high pressure run is complete, the low-and high-pressure data files of the same sample are merged.

The total weight of the shell or shell portion is preferably about 2 percent to about 400 percent of the weight of the core. The total weight of the shell or shell portion is typically from about 5 percent to about 200 percent, e.g. from about 10 percent to about 150 percent of the weight of the core.

Typical shell or shell portion thicknesses which may be employed in this invention are about 20 to about 2000 microns. In certain preferred embodiments, the shell or shell portion has a thickness of less than 800 microns, e.g. about 100 to about 400 microns.

In another embodiment of the invention, the core or portion thereof and/or the shell or portion thereof is made using the thermal cycle molding method and apparatus described in copending U.S. patent application Ser. No. 09/966,497, pages 27-51, the disclosure of which is also incorporated herein by reference. In the thermal cycle molding method and apparatus of U.S. patent application Ser. No. 09/966,497, a thermal cycle molding module having the general configuration shown in FIG. 3 therein is employed. The thermal cycle molding module 200 comprises a rotor 202 around which a plurality of mold units 204 are disposed. The thermal cycle molding module includes a reservoir 206 (see FIG. 4) for holding flowable material to make the core, the shell, a core portion, or a shell portion. In addition, the thermal cycle molding module is provided with a temperature control system for rapidly heating and cooling the mold units. FIGS. 55 and 56 depict such a temperature control system 600.

The dosage form of this invention may be prepared by a method comprising:

(a) admixing the following components:

(i) about 25 to about 40 weight percent of at least one non-aqueous carrier material which has a melting temperature less than about 45 degrees C.,

(ii) about 15 to about 60 weight percent of at least one thermoplastic material which has a melting temperature greater than about 50 degrees C., and

(iii) at least one active ingredient;

(b) providing the admixture into a mold at a temperature in the range of about 0 to 40 degrees C.;

(c) heating the mold and admixture contained therein to a temperature in the range of about 50 to 100 degrees C.; and

(d) cooling the mold and admixture contained therein to a temperature in the range of about 0 to about 25 degrees C.

The edible solid composition, core or portion thereof, or shell or portion thereof of this invention may be prepared by a method comprising:

(a) admixing the following components:

(i) about 25 to about 40 weight percent of at least one non-aqueous carrier material which has a melting temperature less than about 45 degrees C., and

(ii) about 15 to about 60 weight percent of at least one thermoplastic material which has a melting temperature greater than about 50 degrees C.;

(b) providing the admixture into a mold at a temperature in the range of about 0 to 40 degrees C.;

(c) heating the mold and admixture contained therein to a temperature in the range of about 50 to 100 degrees C.; and

(d) cooling the mold and admixture contained therein to a temperature in the range of about 0 to about 25 degrees C.

This invention will be illustrated by the following examples, which are not meant to limit the invention in any way.

### EXAMPLE 1

Dosage forms according to the invention comprising a core within a shell having a first shell portion and a second shell portion were prepared as follows.

The following ingredients were used to make the core (Ketoprofen tablet):

| | | | Weight | Mg/Dosage |
|---|---|---|---|---|
| Ingredient | Trade Name | Manufacturer | % | Form |
| Ketoprofen | | Societa Italiana Med. | 15 | 73.7 |
| | | Scandicci, Reggello, | | |
| | | Italy | | |
| Polyethylene Oxide | Polyox ® WSRN-80 | Union Carbide | 75 | 368.6 |
| (MW 200,000) | | Corporation, Danbury, | | |
| | | CT | | |
| Hydroxypropyl | Methocel E5 | Dow Chemical | 8.5 | 41.8 |
| Methylcellulose | | Company, | | |
| | | Midland, MI | | |
| Magnesium Stearate | | Mallinckrodt Inc., St. | 1.5 | 7.4 |
| | | Louis, MO | | |
| FD&C Blue #1 | | | Trace | |
| | | | Amount | |
| Alcohol USP (dried as | | | | |
| solvent) | | | | |

The Ketoprofen, hydroxypropyl methylcellulose, blue dye and PEO (MW=200,000), were first mixed in a plastic bag for 5 minutes. This powder mixture was added into the (5 qt) bowl of a planetary mixer (Hobart Corp., Dayton, Ohio). The alcohol was added to the 10 powder mixture while mixing at low speed. The ingredients were mixed for 2 minutes. The resulting granulation was removed from the bowl and dried at room temperature for 12 to 16 hours to remove all residual solvent. The granulation was screened through a #20 mesh screen and was put into a plastic bag. Magnesium stearate was added to the dry granules, followed by mixing for 5 minutes to form the granulation blend.

Cores were then prepared by pressing the granulation using a Manesty Beta-press (Thomas Engineering, Inc., Hoffman Estates, Ill.). A round, concave punch and die unit having 0.4375′ diameter was used for compression. Granulation was fed into the cavity of the press and compressed into solid cores.

The shell portion was made using the following ingredients:

| | Trade | | Weight | Mg/Dosage |
|---|---|---|---|---|
| Ingredient | Name | Manufacturer | % | Form |
| Mineral | | Witco Corporation, | 27.4 | 79.3 |
| Oil | | Memphis, TN | | |
| Glyceryl | Compritol | Gattefosse Corporation, | 24.2 | 70.1 |
| Behenate | 888 ATO | Westwood, NJ | | |
| Polyvinyl | | Union Carbide | 48.4 | 140.1 |
| Acetate 40 | | Corporation, Danbury, | | |
| | | CT | | |

The polyvinyl acetate (milled) was added to a beaker containing mineral oil and mixed using a mixer until all powder was dispersed. An agitating speed of 500 rpm was used. Glyceryl behenate was added to the mixture of polyvinyl acetate and mineral oil, which was again mixed until all powder was dispersed. The shell portion material was provided in flowable form.

A thermal cycle molding module as described in copending U.S. application Ser. No. 09/966,497 at pages 27-51, the disclosure of which is incorporated herein by reference, was used to apply the first coating material onto the cores. The thermal cycle molding module was a laboratory scale unit and comprised a single mold made from an upper mold assembly and a lower mold assembly. The lower mold assembly was first cycled to a cold stage at 25° C. for 30 seconds. The coating material was then introduced into a cavity in the lower mold assembly. A core as prepared above was then inserted into the same cavity. The upper mold assembly was then cycled to a cold stage at 25° C. for 30 seconds. The coating material was added to a cavity in the upper mold assembly. The lower and upper mold assemblies were mated and cycled to a hot stage at 85° C. for 3 minute, followed by cycling to a cold stage at 10° C. for 5 minute to harden the coating. The upper and lower mold assemblies were separated and the core coated with the coating was ejected. The “weight gains” of the cores due to the presence of the coating were recorded.

### EXAMPLE 2

The shell portion was made using the following ingredients:

| | Trade | | Weight | Mg/Dosage |
|---|---|---|---|---|
| Ingredient | Name | Manufacturer | % | Form |
| Propylene | | Arco Chemical Co., | 43.5 | 125.9 |
| Glycol | | Newtown Square, PA | | |
| Glyceryl | Compritol | Gattefosse Corporation, | 17.4 | 50.4 |
| Behenate | 888 ATO | Westwood, NJ | | |
| Polyvinyl | | Union Carbide | 39.1 | 113.3 |
| Acetate 40 | | Corporation, Danbury, | | |
| | | CT | | |

The polyvinyl acetate (milled) was added to a beaker containing propylene glycol and mixed using a mixer until all powder was dispersed. An agitating speed of 500 rpm was used. Glyceryl behenate was added to the mixture of polyvinyl acetate and propylene glycol, which was again mixed until all powder was dispersed. The shell portion material was provided in flowable form.

The core (ketoprofen tablet) of Example 1 was coated with the mixture of glyceryl behenate, polyvinyl acetate and propylene glycol. The coating procedure as described in Example 1 was used to prepare the coated tablet.

### EXAMPLE 3

The shell portion was made using the following ingredients:

| | Trade | | Weight | Mg/Dosage |
|---|---|---|---|---|
| Ingredient | Name | Manufacturer | % | Form |
| Propylene | | Arco Chemical Co., | 40 | 115.8 |
| Glycol | | Newtown Square, PA | | |
| Carnauba | | Strahl & Pitsch Inc., West | 25 | 73.4 |
| wax | | Babylon, NY | | |
| Polyvinyl | | Union Carbide | 35 | 101.3 |
| Acetate 40 | | Corporation, Danbury, CT | | |

The polyvinyl acetate (milled) was added to a beaker containing propylene glycol and mixed using a mixer until all powder was dispersed. An agitating speed of 500 rpm was used. Carnauba wax was added to the mixture of polyvinyl acetate and propylene glycol, which was again mixed until all powder was dispersed. The shell portion material was provided in flowable form.

The core (ketoprofen tablet) of Example 1 was coated with the mixture of Carnauba wax, polyvinyl acetate and propylene glycol. The coating procedure as described in Example 1 was used to prepare the coated tablet.

### EXAMPLE 4

The shell portion was made using the following ingredients:

| | Trade | | Weight | Mg/Dosage |
|---|---|---|---|---|
| Ingredient | Name | Manufacturer | % | Form |
| Mineral | | Witco Corporation, | 35 | 95.8 |
| Oil | | Memphis, TN | | |
| Glyceryl | Compritol | Gattefosse Corporation, | 25 | 68.5 |
| Behenate | 888 ATO | Westwood, NJ | | |
| Polycapro- | CAPA | Solvay Interox, Inc., | 40 | 109.5 |
| lactones | 686 | Laporte, TX | | |

The polycaprolactones (milled) was added to a beaker containing mineral oil and mixed using a mixer until all powder was dispersed. An agitating speed of 500 rpm was used. Glyceryl behenate was added to the mixture of polycaprolactones and mineral oil, which was again mixed until all powder was dispersed. The shell portion material was provided in flowable form.

The core (ketoprofen tablet) of Example 1 was coated with the mixture of polycaprolactones, glyceryl behenate and mineral oil. The coating procedure as described in Example 1 was used to prepare the coated tablet.

Although this invention has been illustrated by reference to specific embodiments, it will be apparent to those skilled in the art that various changes and modifications may be made which clearly fall within the scope of the invention.

The invention claimed is:

1. An edible solid composition comprising:
(a) about 25 to about 40 weight percent based on the weight of the edible composition of at least one non-aqueous carrier material which has a melting temperature less than about 45 degrees C.; and
(b) about 15 to about 60 weight percent based on the weight of the edible composition of at least one thermoplastic material which has a melting temperature greater than about 50 degrees C.

2. The composition of claim 1, in which the non-aqueous carrier material is non-volatile.

3. The composition of claim 1, in which the non-aqueous carrier material has a melting point less than about 25 degrees C.

4. The composition of claim 1, in which the non-aqueous carrier material is selected from the group consisting of mineral oil, propylene glycol, glycerin, polyethylene glycol having a molecular weight in the range of about 1000 to about 20,000, vegetable oil, dibutyl sebacate, triethyl citrate, tributyl citrate, triacetin, diethyl phthalate, dibutyl phthalate, dimethyl phthalate, acetyltributyl citrate, acetyltriethyl citrate, ethylene oxide/propylene oxide copolymers, polyoxyethylene alkyl ethers, polyethoxylated castor oil, polyoxyethylenesorbatan fatty acid esters, and combinations thereof.

5. The composition of claim 1, in which the thermoplastic material is selected from the group consisting of polyvinyl acetate, polyalkylene glycols such as polyethylene glycol having a molecular weight in the range of about 1000 to about 20,000, or polyethylene oxide; shellac, polycapractones, polyvinyl alcohol, cetyl alcohol, or combinations thereof.

6. The composition of claim 1, in which the composition additionally comprises up to about 40 weight percent based on the weight of the edible composition of at least one compatibility material for retaining the non-aqueous carrier material in the edible solid composition, wherein the compatibility material is selected from the group consisting of emulsifiers, acrylic polymers, waxes and combinations thereof.

7. The composition of claim 6, in which the compatibility material is selected from the group consisting of camuba wax, beeswax, microcrystalline wax, and combinations thereof.

8. The composition of claim 6, in which the compatibility material is a fatty acid ester, an anionic methacrylic polymer, or a combination thereof.

9. The composition of claim 8, in which the fatty acid ester is selected from the group consisting of glyceryl monostearate, glyceryl palmitostearate, glyceryl behenate, and combinations thereof.

10. The composition of claim 8, in which the anionic methacrylic polymer is an anionic methacrylic copolymer having less than about 35% methacrylic acid units on a molar basis.

11. The composition of claim 1, in which the composition is contained within a core or core portion of a dosage form.

12. The composition of claim 1, in which the composition is contained within a shell or shell portion of a dosage form.

13. A dosage form comprising:
(I) an edible solid composition comprising:
(a) about 25 to about 40 weight percent of at least one non-aqueous carrier material which has a melting temperature less than about 45 degrees C., and
(b) about 15 to about 60 weight percent of at least one thermoplastic material which has a melting temperature greater than about 50 degrees C.; and
(II) at least one active ingredient.

14. The dosage form of claim 13, in which the edible solid composition is contained within a core or core portion of the dosage form.

15. The dosage form of claim 13, in which the edible solid composition is contained within a shell or shell portion of the dosage form.

16. The dosage form of claim 13, in which the non-aqueous carrier material is non-volatile.

17. The dosage form of claim 13, in which the non-aqueous carrier material has a melting point less than about 25 degrees C.

18. The dosage form of claim 13, in which the non-aqueous carrier material is selected from the group consisting of mineral oil, propylene glycol, glycerin, polyethylene glycol having a molecular weight in the range of about 1000 to about 20,000, vegetable oil, dibutyl sebacate, triethyl citrate, tributyl citrate, triacetin, diethyl phthalate, dibutyl phthalate, dimethyl phthalate, acetyltributyl citrate, acetyltriethyl citrate, ethylene oxide/propylene oxide copolymers, polyoxyethylene alkyl ethers, polyethoxylated castor oil, polyoxyethylenesorbatan fatty acid esters, and combinations thereof.

19. The dosage form of claim 13, in which the thermoplastic material is selected from the group consisting of polyvinyl acetate, polyethylene glycol having a molecular weight in the range of about 1000 to about 20,000, shellac, polyethylene oxide, polycapractones and combinations thereof.

20. The dosage form of claim 13, in which the shell additionally comprises up to about 40 weight percent of at least one compatibility material for retaining the non-aqueous carrier material in the core, wherein the compatibility material is selected from the group consisting of emulsifiers, acrylic polymers, waxes and combinations thereof.

21. The dosage form of claim 20, in which the compatibility material is selected from the group consisting of camuba wax, beeswax, microcrystalline wax, and combinations thereof.

22. The dosage form of claim 20, in which the compatibility material is a fatty acid ester, an anionic methacrylic polymer, or a combination thereof.

23. The dosage form of claim 22, in which the fatty acid ester is selected from the group consisting of glyceryl monostearate, glyceryl palmitostearate, glyceryl behenate, and combinations thereof.

24. The dosage form of claim 22, in which the anionic methacrylic polymer is an anionic methacrylic copolymer having less than about 35% methacrylic acid units on a molar basis.

25. An edible solid composition prepared by a process comprising:
(a) admixing the following components:
(i) about 25 to about 40 weight percent based on the weight of the edible solid composition of at least one non-aqueous carrier material which has a melting temperature less than about 45 degrees C., and
(ii) about 15 to about 60 weight percent based on the weight of the edible solid composition of at least one thermoplastic material which has a melting temperature greater than about 50 degrees C.;
(b) providing the admixture into a mold at a temperature in the range of about 0 to about 40 degrees C.;
(c) heating the mold and admixture contained therein to a temperature in the range of about 50 to about 100 degrees C.; and
(d) cooling the mold and admixture contained therein to a temperature in the range of about 0 to about 25 degrees C.

26. The composition of claim 25, in which the non-aqueous carrier material is non-volatile.

27. The composition of claim 25, in which the non-aqueous carrier material has a melting point less than about 25 degrees C.

28. The composition of claim 25, in which the non-aqueous carrier material is selected from the group consisting of mineral oil, propylene glycol, glycerin, polyethylene glycol having a molecular weight in the range of about 1000 to about 20,000, vegetable oil, dibutyl sebacate, triethyl citrate, tributyl citrate, triacetin, diethyl phthalate, dibutyl phthalate, dimethyl phthalate, acetyltributyl citrate, acetyltriethyl citrate, ethylene oxide/propylene oxide copolymers, poiyoxyethylene alkyl ethers, polyethoxylated castor oil, polyoxyethylenesorbatan fatty acid esters, and combinations thereof.

29. The composition of claim 25, in which the thermoplastic material is selected from the group consisting of polyvinyl acetate, polyethylene glycol having a molecular weight in the range of about 1000 to about 20,000, shellac, polyethylene oxide, polycapractones and combinations thereof.

30. The composition of claim 25, in which the admixture additionally comprises up to about 40 weight percent of at least one compatibility material for retaining the non-aqueous carrier material in the edible solid composition, wherein the compatibility material is selected from the group consisting of emulsifiers, acrylic polymers, waxes and combinations thereof.

31. The composition of claim 30, in which the compatibility material is selected from the group consisting of camuba wax, beeswax, microcrystalline wax, and combinations thereof.

32. The composition of claim 30, in which the compatibility material is a fatty acid ester, an anionic methacrylic polymer, or a combination thereof.

33. The composition of claim 32, in which the fatty acid ester is selected from the group consisting of glyceryl monostearate, glyceryl palmitostearate, glyceryl behenate, and combinations thereof.

34. The composition of claim 32, in which the anionic methacrylic polymer is an anionic methacrylic copolymer having less than about 35% methacrylic acid units on a molar basis.

35. The composition of claim 25, in which the edible solid composition is contained within a core or core portion in a dosage form.

36. The composition of claim 25, in which the edible solid composition is contained within a shell or shell portion in a dosage form.

37. The composition of claim 25, in which the edible solid composition is contained within a dosage form.

38. A method for preparing an edible solid composition, wherein the method comprises:
a) admixing the following components:
(i) about 25 to about 40 percent by weight of the edible solid composition of at least one non-aqueous carrier material which has a melting temperature less than about 45 degrees C., and
(ii) about 15 to about 60 percent by weight of the edible solid composition of at least one thermoplastic material which has a melting temperature greater than about 50 degrees C.;
b) providing the admixture into a mold at a temperature in the range of about 0 to 40 degrees C.;
c) heating the mold and admixture contained therein to a temperature in the range of about 50 to 100 degrees C.; and
d) cooling the mold and admixture contained therein to a temperature in the range of about 0 to about 25 degrees C.

39. The method of claim 38, in which the edible solid composition is contained within a shell or shell portion for use in a dosage form.

40. The method of claim 38, in which the edible solid composition is contained within a core or core portion for use in a dosage form.

41. The method of claim 38, in which the edible solid composition is contained within a dosage form.

42. The method of claim 38, in which the admixture additionally comprises up to about 40 weight percent of at least one compatibility material for retaining the non-aqueous carrier material in the edible solid composition, wherein the compatibility material is selected from the group consisting of emulsifiers, acrylic polymers, waxes and combinations thereof.

43. A modified release solid dosage form comprising one or more active ingredients, and an edible solid composition comprising:
a) about 25 to about 40 weight percent based on the weight of the edible solid composition of at least one non-aqueous carrier material which has a melting temperature less than about 45 degrees C.; and
b) about 15 to about 60 weight percent based on the weight of the edible solid composition of at least one thermoplastic material which has a melting temperature greater than about 50 degrees C.

44. The dosage form of claim 43, in which the dosage form comprises a shell, and the edible solid composition is contained in at least a first portion of the shell.

45. The dosage form of claim 44, wherein the edible solid composition functions as a diffusible matrix.

46. The dosage form of claim 44, wherein the edible solid composition functions as an impermeable barrier to the passage of solvent or active ingredient therethrough.

47. The dosage form of claim 43, wherein one or more active ingredients are selected from the group consisting of pharmaceuticals, minerals, vitamins, and nutraceuticals.

48. The dosage form of claim 43, in which the dosage form comprises a core, and the edible solid composition is contained in at least a portion of the core.

49. The dosage form of claim 48, wherein the edible solid composition is in the form of a plurality of particles having an average diameter from about 100 to about 2000 microns.

50. The dosage form of claim 48, wherein the edible solid composition is at least about 90% by weight of the weight of the core.

51. The dosage form of claim 13 or claim 43, wherein one or more active ingredients are released in a controlled, sustained, prolonged, or extended manner upon contacting of the dosage form with a liquid medium.

52. The dosage form of claim 48, wherein the edible solid composition functions as a diffusible matrix.

53. The dosage form of claim 13, wherein the dosage form functions as a diffusible matrix.
